(19) United States
(12) Patent Application Publication
(10) Pub. No.: US 2014/0128270 A1
(43) Pub. Date: May 8, 2014

(54) Method of improving microarray performance by strand elimination

(71) Applicant: Roche Molecular Systems, Inc., Pleasanton, CA (US)

(72) Inventor: AKI NAKAO, SUNNYVALE, CA (US)

(73) Assignee: Roche Molecular Systems, Inc., Pleasanton, CA (US)

(21) Appl. No.: 14/067,746

(22) Filed: Oct. 30, 2013

Related U.S. Application Data
(60) Provisional application No. 61/724,156, filed on Nov. 8, 2012.

(51) Int. Cl. C12Q 1/68

(52) U.S. Cl. CPC C12Q 1/6874; C12Q 1/6886
USPC 506/2; 506/38

(57) ABSTRACT

The invention is a method of determining nucleotide sequence of a target nucleic acid using microarray analysis. Hybridization signals from probe sets corresponding to the sense and anti-sense strands are compared at each nucleotide position. If there is a substantial difference in performance between the two strands, probe sets from a poorly performing stand are eliminated from the sequence determination calculation for a particular nucleotide.

### BACKGROUND OF THE INVENTION

Oligonucleotide microarrays (chips) are an economical way of analyzing multiple nucleic acid targets in one experiment. These arrays are commonly used to analyze multiple genes, for example in a gene expression assay. However, oligonucleotide microarrays are also gaining popularity as an economical and convenient alternative to sequencing in somatic and germline mutation detection assays. Certain genes are well known as somatic mutation and polymorphism hotspots. For such genes, many of the somatic mutations and polymorphisms are associated with a disease or an altered phenotype. For example, multiple mutations in the TP53 and EGFR genes are relevant to cancer. Somatic mutations in TP53 gene are known to cause loss of p53 function, associated with an increase of cancers occurring in various tissues. The TP53 mutation status is also useful for prognosis and predicting response to therapy. Similarly, multiple polymorphisms in CYP450 gene effectively predict the pattern of drug metabolism. Because of the large number of mutations, targeting each mutation with a separate assay becomes impractical. Thus microarrays capable of at once probing multiple mutated base positions (or even every base position within the gene) offer a useful alternative.

A typical microarray (chip) is a collection of microscopic spots each containing millions of nucleic acid probes attached to a solid surface. The probes are capable of hybridizing to the labeled DNA fragments from a sample under suitable conditions. Probe-target hybridization is detected and optionally quantified by detection of a label conjugated to the target molecule. Microarrays as a mutation detection tool have been validated in several systems (reviewed in Schwartz, S., Clinical Utility of Single Nucleotide Polymorphism Arrays (2011) Clin. Lab. Med. 31:581.) Unfortunately, studies involving microarrays report that the sensitivity and specificity of microarrays are not yet ideal compared to existing technologies (see e.g. Zin R., et al., SNP-based arrays complement classic cytogenetics in the detection of chromosomal aberrations in Wilms' tumor (2012) Cancer Genetics 205:80. In practice, it appears that performance of microarrays is not uniform throughout the probed sequence. Some positions within the sequence are subject to error more than others. The use of better mathematical or statistical tools for data analysis that identify such special sites holds the promise of improving sensitivity and specificity of mutation detection microarrays.

### SUMMARY OF THE INVENTION

In some embodiments, the invention is a method of interrogating a sequence of a target nucleic acid having a sense and an anti-sense strands by a microarray analysis comprising a sequence determination computation, comprising omitting from the computation a signal from one of the sense and anti-sense strands for one or more nucleotide positions in the target nucleic acid sequence. In variations of this embodiment, omitting the signal from one of the sense and anti-sense strands at a nucleotide position comprises the steps of using a plurality of microarrays, measuring hybridization signals at the nucleotide position using one or more probe sets for each of the sense and the anti-sense strands; for each probe set, determining base discrimination ability by comparing the hybridization signals within each probe set; for each nucleotide position, computing discrimination ability for sense and antisense strand separately using the computed discrimination ability from each of the probe sets; for each nucleotide position, comparing the computed discrimination ability between the sense and the anti-sense strands; omitting the signal from the strand with lower base discrimination ability. In variations of this embodiment, the base discrimination is measured using Formula 1. In further variations of this embodiment, the discrimination ability for sense and antisense strand is computed as a percentile of the discrimination ability for probe sets in the strand at the base position. In yet further variations of this embodiment, the discrimination ability between sense and antisense starnd is compared using Formula 3.

In other embodiments, the invention is a method of detecting the presence or absence of a target nucleic acid having a sense and an anti-sense strands in a test sample using a microarray analysis including a sequence determination or mutation detection computation, comprising omitting from the computation a signal from one of the sense and anti-sense strands for one or more nucleotide positions in the target nucleic acid sequence. In variations of this embodiment, omitting the signal from one of the sense and anti-sense strands at a nucleotide position comprises the steps of: using a plurality of microarrays, measuring hybridization signals at the nucleotide position using one or more probe sets for each of the sense and the anti-sense strands; for each probe set, determining base discrimination by comparing the hybridization signals within each probe set; for each nucleotide position, computing discrimination ability for sense and antisense strand separately using discrimination ability from each of the probe sets; for each nucleotide position, comparing discrimination ability between the sense and the anti-sense strands; omitting the signal from a strand with lower base discrimination ability. In variations of this embodiment, the base discrimination is measured using Formula 1. In further variations of this embodiment, the discrimination ability of the sense and anti-sense strand is computed as a percentile of the discrimination ability for probe sets in the strand at the base position measured using a plurality of microarrays.

In yet another embodiment, the invention is a computer readable medium including code for controlling one or more processors to detect the presence or absence of a target nucleic acid having a sense and an anti-sense strands in a test sample using a microarray analysis that includes a sequence determination or mutation detection computation, comprising omitting from the computation a signal from one of the sense and anti-sense strands for one or more nucleotide positions in the target nucleic acid sequence. In variations of this embodiment, the computer readable medium comprises a code controlling the steps of: using a plurality of microarrays, measuring hybridization signals at the nucleotide position using one or more probe sets for each of the sense and the anti-sense strands; for each probe set, determining base discrimination ability by comparing the hybridization signals within each probe set; for each nucleotide position, computing discrimination ability for sense and antisense strand separately using discrimination ability from each of the probe sets; for each nucleotide position, comparing discrimination ability between the sense and the anti-sense strands; omitting the signal from a strand with lower base discrimination ability.

In yet another embodiment, the invention is a system for detecting a target nucleic acid in a test sample comprising: a data acquisition module configured to acquire hybridization data from a microarray; a data processing unit configured to process the data to determine a target nucleotide sequence by omitting the signal from one of the sense and anti-sense strands at one or more nucleotide positions in the target sequence via the steps of: using a plurality of microarrays, measuring hybridization signals at the nucleotide position using one or more probe sets for each of the sense and the anti-sense strands; for each probe set, determining base discrimination ability by comparing the hybridization signals within each probe set; for each nucleotide position, computing discrimination ability for sense and antisense strand separately using discrimination ability from each of the probe sets; for each nucleotide position, comparing discrimination ability between the sense and the anti-sense strands; omitting the signal from a strand with lower base discrimination ability; and a display module configured to display the data produced by the data processing unit.

In yet another embodiment, the invention is a method of detecting the presence or absence of a mutation in the p53 gene in a test sample using a microarray analysis including a mutation detection computation, comprising omitting from the computation a signal from one of the sense and anti-sense strands for one or more nucleotide positions in the target nucleic acid sequence. In variations of this embodiment, the nucleotide positions are selected from codon 273, position 1 and codon 220, position 2 within the p53 gene.

### BRIEF DESCRIPTION OF THE FIGURES

FIG. 1. Selecting strands for elimination by comparing discriminating ability of sense and antisense probes using Q₇₅ value.

### DETAILED DESCRIPTION OF THE INVENTION

### Definitions

The terms “nucleic acid,” and “oligonucleotide” refer to target sequences and probes. The terms are not limited by length and are generic to linear polymers of deoxyribonucleotides (single-stranded or double-stranded DNA), ribonucleotides (RNA), and any other N-glycoside of a purine or pyrimidine base, including adenosine, guanosine, cytidine, thymidine and uridine and modifications of these bases.

The term “probe” refers to an oligonucleotide that selectively hybridizes to a target nucleic acid under suitable conditions.

The term “probe set” refers to a group of two or more probes in a microarray designed to interrogate the mutation status in the same base position within a target sequence. A typical probe set contains five or more different probes; one for hybridizing to wildtype DNA sequence from a sample, three probes for three possible single-base substitutions, and one probe for detecting a single base pair deletion at the interrogating position. Additional probes may be added, e.g. the sixth probe can be included to detect two base-pair deletion.

The term “target site” or “target base position” refers to the base position in the target nucleic acid that is interrogated by a probe in the probe set. Multiple overlapping probes within the probe sets may interrogate the same target site.

The terms “target sequence” or “target” refer to a region of a nucleic acid sequence that is to be analyzed.

The term “sample” refers to any composition containing or presumed to contain nucleic acid. This includes a sample of tissue or fluid isolated from an individual for example, skin, plasma, serum, spinal fluid, lymph fluid, synovial fluid, urine, tears, blood cells, organs, bone marrow and tumors, including the fresh or fresh-frozen tissue and formalin-fixed paraffin embedded tissue (FFPET), and also to samples of in vitro cultures established from cells taken from an individual, and nucleic acids isolated therefrom.

The term “training set” refers to a set of samples used to build data analysis algorithms including statistical models.

The term “training data set” refers to a set of microarray data obtained from the training set. A training data set may be a set of microarray data obtained from samples where the sequences are known. For example, the training data set may be used to build a statistical model to determine the mutation status and to identify specific nucleotide positions where the intensity patterns are consistently different from the rest within the sequence and modify the mutation detection algorithm.

The terms “testing set” refers to a set of one or more samples used to verify the mutation detection algorithm built using the training set.

The term “testing data set” refers to a set of microarray data obtained from the testing set. A testing data set may be a set of microarray data obtained from samples where the sequences (mutation status) are known. For example, the testing data set may be used to verify effective mutation detection by the algorithm built based on the data from the training data set.

The term “test sample” refers to a sample used to generate testing dataset.

The term “re-sequencing by microarray” or “mutation detection by microarray” are used interchangeably to refer to a method of mutation detection within the target sequence by detecting and analyzing hybridization signals from multiple probe sets arranged on a microarray, each set corresponding to a nucleotide position within the sense and the anti-sense strand of the target sequence, hybridized to the labeled nucleic acid fragments present in a sample. Typically, re-sequencing by microarray comprises an algorithm that detects mutated nucleic acid in a background of wild-type nucleic acid utilizing the hybridization signals from multiple probe sets. The term “re-sequencing” encompasses determination of mutation status in the entire sequence of the target nucleic acid as well as determination of fewer than all nucleotides, e.g. only one or several selected nucleotides in the target nucleic acid that are known sites of mutations.

The present invention comprises a method of improving the accuracy of re-sequencing and mutation detection microarrays. A microarray is a collection of nucleic acid probes designed to detect mutations in a background of wild-type nucleic acid sequences. That is, under suitable hybridization conditions, the probes would preferentially hybridize only to the target sequence present in the sample genome. Each probe set is designed to detect three possible single base pair substitutions and a single base pair deletion for a particular nucleotide position within the target sequence. Several overlapping probe sets with different probe length may be designed to detect an individual mutation. A microarray may contain probe sets designed to detect mutations in some or all of the nucleotides in the target sequence. Furthermore, a microarray may contain probe sets corresponding to nucleotides on both strands of the target sequence. Depending on the number of nucleotides to be interrogated, an array may contain thousands or even millions of probe sets (see Schena, M. (ed.), Microarray Biochip Technology (2000) Eaton Pub. Co. (Westborough, Mass.).

Each probe set typically contains five probes: four for each of the possible nucleotides at the particular position within the target sequence and one probe for a deletion of the nucleotide at that position. Upon incubation under appropriate protocols, the probes emit detectable signals. Ideally, one of the five probes within the probeset emits a much greater signal than the other four if the interrogating position is wild-type, and two of the five probes emit greater signals than the other three when the sample contains mutated DNA in addition to the wild-type DNA. Since most of the somatic mutations are heterozygous, and a typical clinical sample contains both cancer and non-cancer cells, wild-type signals are present for most of the cases. The detector registers the signal associated with a particular probe for a particular strand for a particular nucleotide at that position within the target sequence.

The software algorithm currently used for making nucleotide calls, examines the data from both sense and anti-sense probe sets for each position. Only when both sense and anti-sense signals are in a certain agreement on a mutation in a particular position within the target sequence, the software makes the mutation call for the nucleotide at that position.

The present invention is a method of improving mutation detection or re-sequencing by microarray analysis comprising omitting a signal from one of the two complementary strands from a mutation detection algorithm for one or more nucleotide positions in the target sequence.

The inventors observed that each array trends towards a number of trouble spots within the target sequence. These trouble spots become apparent when the array is tested with multiple samples of different origin and different quality of nucleic acids to be tested. The array is consistently unable to make correct calls for certain positions within the target sequence. Aiming to eliminate or reduce the number of such missed calls, the inventors investigated the source of error at the trouble spots. Surprisingly, it was discovered that for some positions within the target sequence, there was a dramatic difference in performance between the sense and antisense probe sets. Accordingly, the inventors devised a mathematical method to identify such nucleotide positions and eliminate the data obtained from the poorly performing strand from computation. The strands with poor performance are identified according to the teaching of the invention. It is noted that where both sense and antisense probes perform poorly; probes from neither strand are eliminated.

In one embodiment, the invention is a method comprising obtaining a microarray data set by hybridizing the labeled and fragmented nucleic acids from a sample to an oligonucleotide microarray and obtaining the hybridization data and converting the hybridization data into the probe intensities and analyzing the probe intensity data to extract biologically meaningful information such as nucleic acid sequence or presence of mutations. Oligonucleotide microarrays may be custom made as described in Schena, M. (ed.), Microarray Biochip Technology (2000) or obtained from commercial suppliers such as e.g., Affymetrix (Santa Clara, Cal.), NimbleGen (Madison, Wisc.), and Agilent Tech. (Santa Clara, Cal.). The optimal conditions for generating high quality microarray data, such as sample preparation, amplification, fragmentation and labeling of nucleic acids, hybridization and washing may be obtained from the manufacturers of microarrays, or determined empirically by one skilled in the art of nucleic acid chemistry. To determine the sample sequence or identify mutations in a sample, the microarray hybridization data may be analyzed by any microarray analysis algorithm known in the art, e.g., Microarray Suite (MAS), or Gene Chip Operating System (GCOS) (Affymetrix, Santa Clara, Cal.).

In this embodiment, multiple microarray experiments are conducted to obtain microarray data sets and identify persistent trouble spots within the target nucleic acid sequence. Optionally multiple experiments are conducted with target nucleic acids isolated from various sources to identify trouble spots that are independent of the source and quality of the nucleic acid in the sample. In some variations of this embodiment, prior to analyzing a test sample, one or more training samples are analyzed to identify trouble spots in the training data sets. The training samples may comprise mixtures of target nucleic acids with and without mutations to simulate patient samples that contain mutant and non-mutant cells.

In some embodiments of the invention, the trouble spots in the microarray data sets are further analyzed to determine whether the probe sets targeting one strand or both strands consistently fail to make the call. In variations of this embodiment, the microarray data sets are analyzed to determine whether the probe sets failing to make the mutation call at the trouble spot are prone to non-specific hybridization.

In some embodiments, the invention comprises a method of identifying poorly performing strands to be eliminated from the sequence determination or mutation detection computation at certain nucleotide positions within the target sequence. To identify whether one strand should be eliminated at a nucleotide position, the following steps may be performed using probe hybridization data for the nucleotide position:
(1) calculate discriminating ability of each probe set;
(2) calculate a percentile of discriminating ability of all probe sets for sense and antisense strands separately for each of the target nucleotide positions;
(3) determine a desired percentile of the values obtained in step (2) across multiple microarrays;
(4) determine the difference between the discriminating ability at the given percentiles obtained in step (3) between the sense and the anti-sense strands;
(5) where the difference obtained in step (4) is substantial or falls above a threshold, eliminate from the mutation detection or re-sequencing computation the strand with the lower discriminating ability.

Any formula for calculating discriminating ability of a probe set may be used. Seo, et. al, Bioinformatics, Vol. 20, No. 16 2534-2544, 2004). In some embodiments, the discrimination ability may be determined by calculating discrimination ratio of mismatch probe (DR_MM). DR_MM for each probe set (DR_MMₛ) is determined according to Formula 1:

DR_{—}MMₛ={(PM−max(MMᵢ))/(PM+max(MMᵢ)),i=1:3} Formula 1

PM—probe intensity of the perfectly matched probe, which is designed to hybridize to the wild-type sequence for the base position

MMᵢ—probe intensity of one of the three mismatched probe, which is designed to hybridize to a single base pair substitution

max(MM)—the maximum probe intensity among the three mismatched probes in the probe set

Any percentile may be used to compute discriminating ability of a strand at a base position using the DR_MM calculated for each probes set, for example, 50^{th} (median), 55^{th}, 60^{th}, 70^{th}, 75^{th} (quartile), 80^{th} or 90^{th} percentiles or any percentiles falling between those numbers. In some embodiments, 50^{th} (median) is used.

Any percentile may be used to identify a poorly performing strand based on the performance of a strand at a base position across multiple microarrays, for example, 55^{th}, 60^{th}, 70^{th}, 80^{th} or 90^{th} percentiles may be used or any percentile falling between those numbers. In some embodiments, the 75^{th} percentile (Q₇₅) is used.

Any threshold for the difference between the percentiles of the sense and anti-sense strands may be used for strand elimination. In some embodiments, the threshold is set according to Formula 2:

Q₇₅ᵢ<Q₇₅ⱼ−T Formula 2

T is the threshold

Q₇₅ᵢ is the Q₇₅ value of a strand i to be eliminated;

Q₇₅ᵢ is the Q₇₅ value of the complementary strand j.

In some embodiments, the threshold equals 0.13 (T=0.13).

In some embodiments, the chosen value of the threshold depends on relative performance of the strands. When discriminating ability of one strand is poor and the difference with the other strand exceed the threshold, the poor strand is eliminated. When discriminating ability is moderately poor, the strand is eliminated only if the other strand performs significantly better, i.e. Q₇₅ is substantially higher than the threshold. If both strands perform poorly, no strand is eliminated. In some embodiments, extremely poor performance is defined as Q₇₅<0.151 and moderately poor performance is defined as 0.151≦Q₇₅<0.3. A threshold value for poor performance (PT) may be empirically determined.

In some embodiments, the threshold may be set according to Formula 3:

(1) Q₇₅ᵢ<Q₇₅ⱼ−T, for Q₇₅ᵢ<PT

(2) Q₇₅ᵢ<A(Q₇₅ⱼ−B)²+PT, for Q₇₅ᵢ≧PT Formula 3

T is the threshold for the difference between the strands;

PT is the threshold for poor performance

A and B are empirically determined

Q₇₅ᵢ is the Q₇₅ value of a strand i to be eliminated;

Q₇₅ⱼ is the Q₇₅ value of the complementary strand j.

In some embodiments, the threshold is 0.13 (T=0.13), the threshold for poor performance is 0.151 (PT=0.151), A=0.42227 and B=0.281.

In some embodiments, the invention comprises a data analysis algorithm for microarrays that are designed to detect mutations in a target nucleotide sequence by omitting the signal from one of the sense and anti-sense strands at one or more nucleotide positions in the target sequence via the steps of: using multiple microarrays, measuring hybridization signals (e.g., probe intensities) at the nucleotide position using one or more probe sets for each of the sense and the anti-sense strands; for each probe set, computing base discrimination ability within each probe set; calculating a percentile of discriminating ability of all probe sets for sense and antisense strands separately for each of the target nucleotide positions; determining a desired percentile of the discriminating ability values across multiple microarrays; determining the difference between the discriminating ability at the given percentiles between the sense and the anti-sense strands; where the difference is substantial or falls above a threshold, eliminating from the mutation detection or re-sequencing computation the strand with the lower discriminating ability.

In some embodiments, the invention comprises a data analysis algorithm for microarrays that are designed to detect mutations in the TP53 gene in a sample by omitting the signal from one of the sense and anti-sense strands at one or more nucleotide positions in the target sequence via the steps of using multiple microarrays, measuring hybridization signals (e.g., probe intensities) at the nucleotide position using one or more probe sets for each of the sense and the anti-sense strands; for each probe set, computing base discrimination ability using the signals within each probe set; calculating a percentile of discriminating ability of all probe sets for sense and antisense strands separately for each of the target nucleotide positions; determining a desired percentile of the discriminating ability values across multiple microarrays; determining the difference between the discriminating ability at the given percentiles between the sense and the anti-sense strands; where the difference is substantial or falls above a threshold, eliminating from the mutation detection or re-sequencing computation the strand with the lower discriminating ability. These mutations are associated with development and progression of certain human cancers. See e.g. Freed-Pastor, W. et al. (2004). “Mutant p53: one name, many proteins” (2012) Genes Dev. 26:1268.

Examples and figures below illustrate applications of the method of the present invention to detecting mutations in the human TP53 (p53) gene.

In some embodiments, the invention is a computer readable medium including code for controlling one or more processors to determine a target nucleotide sequence by omitting the signal from one of the sense and anti-sense strands at one or more nucleotide positions in the target sequence via the steps of: using multiple microarrays, measuring hybridization signals (e.g., probe intensities) at the nucleotide position using one or more probe sets for each of the sense and the anti-sense strands; computing base discrimination ability using the signals within each probe set; calculating a percentile of discriminating ability of all probe sets for sense and antisense strands separately for each of the target nucleotide positions; determining a desired percentile of the discriminating ability values across multiple microarrays; determining the difference between the discriminating ability at the given percentiles between the sense and the anti-sense strands; where the difference is substantial or falls above a threshold, eliminating from the mutation detection or re-sequencing computation the strand with the lower discriminating ability.

In some embodiments, the invention is a system for determining a target nucleotide sequence in a test sample according to the present invention comprising: a data acquisition module configured to acquire a data set from nucleotide microarrays, the data set containing data from the sense and the anti-sense strands; a data processing unit configured to process the data to determine a target nucleotide sequence by omitting the signal from one of the sense and anti-sense strands at one or more nucleotide positions in the target sequence via the steps of: using multiple microarrays, measuring hybridization signals (e.g., probe intensities) at the nucleotide position using one or more probe sets for each of the sense and the anti-sense strands; for each probe set, computing base discrimination ability within the probe set; calculating a percentile of discriminating ability of all probe sets for sense and antisense strands separately for each of the target nucleotide positions; determining a desired percentile of the discriminating ability values across multiple microarrays; determining the difference between the discriminating ability at the given percentiles between the sense and the anti-sense strands; where the difference is substantial or falls above a threshold, eliminating from the mutation detection or re-sequencing computation the strand with the lower discriminating ability; and a display module configured to display the data produced by the data processing unit.

### EXAMPLES

### Example 1

### Identifying Trouble Spots within the p53 Gene in AMPLICHIP® p53

Feasibility studies of AMPLICHIP® p53 (Roche Molecular Diagnostics, Indianapolis, Ind.) to detect mutations in the p53 gene (TP53) showed that detection ability of the following two mutations is not satisfactory: (1) 220_{—}2 (codon 220, 2^{nd} position) A>G, and (2) 273_{—}1 (codon 273, 1^{st} position) C>T. These are among the top six most prevalent mutations found in ovarian cancer.

To confirm the trouble spots, a training set of samples simulating patient samples were prepared as described in Table 1. The samples consisted of mixtures of mutant and wild-type DNA. Mutant DNA was obtained from a cell line whose mutant status at codon 273 was confirmed prior to the study. The mutant DNA was diluted with a p53-wild-type cell line DNA.

TABLE 1
Samples used to evaluate 273_1 C > T mutation detection ability

| | | Base | % mutant |
|---|---|---|---|
| Sample | Codon | change | DNA |
| 1 | 273_1 | C > T | 20 |
| 1 | 273_1 | C > T | 25 |
| 1 | 273_1 | C > T | 33 |
| 2 | 273_1 | C > T | 20 |
| 2 | 273_1 | C > T | 25 |
| 2 | 273_1 | C > T | 33 |

### Example 2

### Investigation of the Nature of the Trouble Spots

A series of analyses of microarray data revealed the following trends for all chips that failed to call the A>G mutation at codon 220_{—}2:
1) The probe set targeting the sense strand calls the A>G mutation but the antisense probe set calls wild type;
2) High amount of cross-hybridization for all probes is observed for the antisense probe sets;
3) In silico analysis of probes in the antisense probe set supports high amount of cross-hybridization.

A series of analyses of microarray data using cell line and clinical samples revealed the following trends for all chips that failed to call the C>T mutation at codon 273_{—}1:
1) The probe set targeting the sense strand calls the C>T mutation but the antisense probe set calls wild type;
2) High amount of cross-hybridization for some probes is observed for the antisense probe set.

### Example 3

### Algorithm Modification by Strand Elimination

A total of 123 reference chips (microarrays) were used (AMPLICHIP® p53 (Roche Molecular Diagnostics, Indianapolis, Ind.)). A reference chip is hybridized with a wild-type cell line and the probe intensities are is used as a baseline to detect mutation in a clinical sample. First, the median of DR_MM (discrimination ratio of mismatch probes) was calculated for each probe set in a base position on each strand for each chip according to Formula 1. DR_MM is a good measure to evaluate the amount of non-specific hybridization. Then, the 75^{th} percentile value (Q₇₅) across the 123 chips was calculated per base position per strand. A very low Q₇₅ value means that the discrimination ability of the probe sets on the strand of the base position is quite poor 75% of the time. Using the Q₇₅ values for sense and antisense strands at the same nucleotide position, the strands were eliminated according to Formula 3 with the following values:

(1) Q₇₅ᵢ<Q₇₅ⱼ−0.130, for Q₇₅ᵢ<0.151

(2) Q₇₅ᵢ<0.4227(Q₇₅ⱼ−0.281)²+0.151, for Q₇₅ᵢ≧0.151
Q₇₅ᵢ is the Q₇₅ value of a strand i to be eliminated;
Q₇₅ⱼ is the Q₇₅ value of the complementary strand j

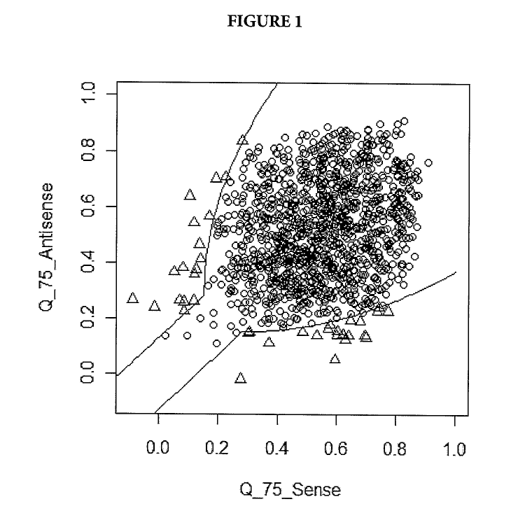

A total of 39 strands were identified and eliminated, i.e. not used for making the mutation calls. They are shown as triangle data points in FIG. 1 and summarized in Table 1.

FIG. 1 illustrates comparison of discriminating ability of sense and antisense probes using Q₇₅ value calculated using 123 chips hybridized with p53 wild-type cell line DNA. The axes represent the Q₇₅ value for each sense and anti-sense strands. Each data point represents one of the 1240 interrogated base positions. The two lines enclose the data points for which no strand is eliminated (circle data points in FIG. 1). The data points fall within three general categories. In the first category, (most cases) the Q₇₅ values for the sense and the anti-sense strands are comparable. These cases fall within the enclosure and not far from the diagonal (Q₇₅_{_{—}}Antisense=Q₇₅_{_{—}}Sense) line on FIG. 1. In the second category, the data points have different Q₇₅ values between sense and anti-sense strands but both values are still reasonably high, (e.g., above 0.2), representing acceptable discrimination ability for the corresponding base position. These data points also fall within the enclosure on FIG. 1. In the third category, the Q₇₅ values are quite different between sense and antisense strands, representing large difference in discriminating ability between the strands, and one strand has quite low Q₇₅, indicating poor discriminating ability. These data points fall outside the enclosure on FIG. 1. For the base positions corresponding to data points within the third category, the worse preforming strand (lower Q₇₅) is eliminated from the mutation calling computation.

In Table 2, “Codon” indicates the codon number and position of the nucleotide within the codon; “Wt” indicates the nucleotide in the wild type sequence; “S” indicates sense strands and “AS” indicates anti-sense strands; “Q75_S” and “Q75_AS” indicate Q₇₅ for the sense and the anti-sense strand respectively; and “abs(Diff75)” indicates the absolute value of the differences between the sense and the anti-sense strands.

TABLE 1
Selecting strands for elimination from
mutation calling computation

| | | | | | Strand |
|---|---|---|---|---|---|
| Codon | Wt | Q75_S | Q75_AS | abs(Diff75) | To Eliminate |
| 138_1 | G | 0.0808 | 0.3808 | 0.3000 | S |
| 144_3 | G | 0.1902 | 0.7001 | 0.5099 | S |
| 151_2 | C | 0.0827 | 0.2584 | 0.1757 | S |
| 151_3 | C | 0.0691 | 0.2647 | 0.1956 | S |
| 152_1 | C | −0.0894 | 0.2665 | 0.3559 | S |
| 157_1 | G | 0.1184 | 0.2616 | 0.1433 | S |
| 158_2 | G | 0.1409 | 0.4131 | 0.2722 | S |
| 159_1 | G | 0.0496 | 0.3657 | 0.3162 | S |
| 160_3 | G | 0.0857 | 0.2257 | 0.1401 | S |
| 161_1 | G | −0.0155 | 0.2378 | 0.2532 | S |
| 180_3 | G | 0.1682 | 0.5666 | 0.3984 | S |
| 226_2 | G | 0.2802 | 0.8360 | 0.5558 | S |
| 248_3 | G | 0.2241 | 0.7074 | 0.4834 | S |
| 249_1 | A | 0.1243 | 0.3757 | 0.2514 | S |
| 249_3 | G | 0.1164 | 0.5409 | 0.4245 | S |
| 282_3 | G | 0.1350 | 0.4658 | 0.3308 | S |
| 336_3 | G | 0.1194 | 0.3569 | 0.2375 | S |
| 347_1 | G | 0.1017 | 0.6385 | 0.5368 | S |
| 12_1 | C | 0.5775 | 0.1767 | 0.4008 | AS |
| 72_3 | C | 0.6312 | 0.1219 | 0.5093 | AS |
| 74_2 | C | 0.5724 | 0.1617 | 0.4108 | AS |
| 110_3 | T | 0.6019 | 0.1390 | 0.4630 | AS |
| 138_2 | C | 0.6415 | 0.1386 | 0.5029 | AS |
| 150_1 | A | 0.6215 | 0.1428 | 0.4787 | AS |
| 158_3 | C | 0.5340 | 0.1379 | 0.3961 | AS |
| 161_2 | C | 0.5952 | 0.0528 | 0.5424 | AS |
| 175_1 | C | 0.6036 | 0.1544 | 0.4492 | AS |
| 220_1 | T | 0.3029 | 0.1460 | 0.1569 | AS |
| 220_2 | A | 0.3063 | 0.1507 | 0.1557 | AS |
| 273_1 | C | 0.7451 | 0.2410 | 0.5041 | AS |
| 274_2 | T | 0.4855 | 0.1507 | 0.3348 | AS |
| 276_3 | C | 0.7741 | 0.2275 | 0.5466 | AS |
| 299_1 | C | 0.6799 | 0.1919 | 0.4880 | AS |
| 318_3 | A | 0.7385 | 0.2273 | 0.5112 | AS |
| 337_1 | C | 0.2766 | −0.0205 | 0.2971 | AS |
| 337_3 | C | 0.3730 | 0.1116 | 0.2614 | AS |
| 347_2 | C | 0.6472 | 0.1949 | 0.4523 | AS |
| 355_2 | C | 0.7003 | 0.1325 | 0.5678 | AS |
| 390_1 | C | 0.6971 | 0.1379 | 0.5592 | AS |

Eliminating the strands from the mutation detection computation resulted in improved mutation detection ability of a re-sequencing microarray (AMPLICHIP® p53 Test) without compromising specificity. Examples of some datasets are shown in Table 3.

TABLE 3
Mutation detection ability of the AMPLICHIP ® Microarray
with and without strand elimination

| | Algorithm Ver 4.0.1 | | Algorithm Ver 4.0.4 | |
|---|---|---|---|---|
| | Mutations detected | | Mutations detected | |
| | without strand | | with strand | |
| Dataset/Study Name | elimination | | elimination | |
| Dataset 1, clinical | 94.3% | (33^{b}/35^{a}) | 97.1% | (34/35) |
| samples (n = 51) | | | | |
| Dataset 2, clinical | 90.9% | (30/33) | 97.0% | (32/33) |
| samples (n = 60) | | | | |
| Clinical samples and | 96.5% | (109/113) | 100.0% | (113/113) |
| cell lines (n = 113) | | | | |
| Cell line containing | 0.0% | (0/12) | 100.0% | (12/12) |
| 273_1 C > T with low | | | | |
| 20-33 mt % (n = 12) | | | | |
| Algorithm test dataset, | 89.6% | (63/67) | 94.0% | (63/67) |
| tumor >=50%, clinical | | | | |
| samples (n = 184) | | | | |
| Clinical samples, | 91.1% | (144/158) | 94.3% | (149/158) |
| FFPE^{c} and FF^{d} (n = 152) | | | | |

^{a}number of mutations already confirmed prior to this study
^{b}number of mutations AMPLICHIP ® p53 correctly called out
^{c}Formalin-Fixed Paraffin Embedded
^{d}Fresh Frozen

While the invention has been described in detail with reference to specific examples, it will be apparent to one skilled in the art that various modifications can be made within the scope of this invention. Thus the scope of the invention should not be limited by the examples described herein, but by the claims presented below.

What is claimed is:

1. A method of interrogating a sequence of a target nucleic acid having a sense and an anti-sense strands by a microarray analysis comprising a sequence determination computation, comprising omitting from the computation a signal from one of the sense and anti-sense strands for one or more nucleotide positions in the target nucleic acid sequence.

2. The method of claim 1, wherein omitting the signal from one of the sense and anti-sense strands at a nucleotide position comprises the steps of:
a. using a plurality of microarrays, measuring hybridization signals at the nucleotide position using one or more probe sets for each of the sense and the anti-sense strands;
b. for each probe set, determining base discrimination ability by comparing the hybridization signals within each probe set;
c. for each nucleotide position, computing discrimination ability for sense and antisense strand separately using discrimination ability from each of the probe sets determined in step (b);
d. for each nucleotide position, comparing discrimination ability between the sense and the anti-sense strands computed in step (c);
e. omitting the signal from the strand with lower base discrimination ability identified in step (d).

3. The method of claim 2, further comprising, in step (d), determining whether the difference in base discrimination falls above a threshold value and in step (e), omitting the signal from a strand corresponding to probe sets with lower base discrimination if the difference falls above the threshold value.

4. The method of claim 2, wherein the probe intensities in step (a) are measured using a plurality of microarrays contacted with a single sample.

5. The method of claim 2, wherein the hybridization signal in step (a) is measured using a plurality of microarrays contacted with a plurality of samples.

6. The method of claim 2, wherein the base discrimination in step (b) is measured using Formula 1.

7. The method of claim 2, wherein in step (c), discrimination ability for sense and antisense strand is computed as a percentile of the values obtained in step (b) for probe sets in the strand at the base position.

8. The method of claim 2, wherein in step (d), discrimination ability between sense and antisense computed in step (c) is compared using Formula 3.

9. The method of claim 7, wherein the percentile is between 60 and 90%.

10. The method of claim 7, wherein the percentile is the third quartile (75%).

11. A method of detecting the presence or absence of a target nucleic acid having a sense and an anti-sense strands in a test sample using a microarray analysis including a sequence determination or mutation detection computation, comprising omitting from the computation a signal from one of the sense and anti-sense strands for one or more nucleotide positions in the target nucleic acid sequence.

12. The method of claim 11, wherein omitting the signal from one of the sense and anti-sense strands at a nucleotide position comprises the steps of:
a. using a plurality of microarrays, measuring hybridization signals at the nucleotide position using one or more probe sets for each of the sense and the anti-sense strands;
b. for each probe set, determining base discrimination by comparing the hybridization signals within each probe set;
c. for each nucleotide position, computing discrimination ability for sense and antisense strand separately using discrimination ability from each of the probe sets determined in step (b);
d. for each nucleotide position, comparing discrimination ability between the sense and the anti-sense strands computed in step (c);
e. omitting the signal from a strand with lower base discrimination ability identified in step (d).

13. The method of claim 11, further comprising, in step (d), determining whether the difference in base discrimination falls above a threshold value and in step (e), omitting the signal from a strand corresponding to probe sets with lower base discrimination if the difference falls above the threshold value.

14. The method of claim 11, wherein the base discrimination in step (b) is measured using Formula 1.

15. The method of claim 11, wherein the discrimination ability of the sense and anti-sense strand in step (c) is computed as a percentile of the values obtained in step (b) for probe sets in the strand at the base position measured using a plurality of microarrays.

16. A computer readable medium including code for controlling one or more processors to detect the presence or absence of a target nucleic acid having a sense and an anti-sense strands in a test sample using a microarray analysis that includes a sequence determination or mutation detection computation, comprising omitting from the computation a signal from one of the sense and anti-sense strands for one or more nucleotide positions in the target nucleic acid sequence.

17. A computer readable medium of claim 15 comprising the code controlling the steps of
a. using a plurality of microarrays, measuring hybridization signals at the nucleotide position using one or more probe sets for each of the sense and the anti-sense strands;
b. for each probe set, determining base discrimination ability by comparing the hybridization signals within each probe set;
c. for each nucleotide position, computing discrimination ability for sense and antisense strand separately using discrimination ability from each of the probe sets determined in step (b);
d. for each nucleotide position, comparing discrimination ability between the sense and the anti-sense strands computed in step (c);
e. omitting the signal from a strand with lower base discrimination ability identified in step (d).

18. A system for detecting a target nucleic acid in a test sample comprising:
a. a data acquisition module configured to acquire hybridization data from a microarray;
b. a data processing unit configured to process the data to determine a target nucleotide sequence by omitting the signal from one of the sense and anti-sense strands at one or more nucleotide positions in the target sequence via the steps of using a plurality of microarrays, measuring hybridization signals at the nucleotide position using one or more probe sets for each of the sense and the anti-sense strands; for each probe set, determining base discrimination ability by comparing the hybridization signals within each probe set; for each nucleotide position, computing discrimination ability for sense and antisense strand separately using discrimination ability from each of the probe sets; for each nucleotide position, comparing discrimination ability between the sense and the anti-sense strands; omitting the signal from a strand with lower base discrimination ability.
c. a display module configured to display the data produced by the data processing unit.

19. The system of claim 18, wherein comparing the base discrimination between the probe sets corresponding to the sense and the anti-sense strands is performed by comparing a percentile of the base discrimination from sense and antisense strands obtained from the plurality of microarrays.

20. A method of detecting the presence or absence of a mutation in the p53 gene in a test sample using a microarray analysis including a mutation detection computation, comprising omitting from the computation a signal from one of the sense and anti-sense strands for one or more nucleotide positions in the target nucleic acid sequence.
